(19) United States
(12) Patent Application Publication
(10) Pub. No.: US 2004/0259772 A1
(43) Pub. Date: Dec. 23, 2004

(54) Use of a histone deacetylase inhibitor to increase the entry of an adenoviral agent into a cell

(76) Inventors: Antonio Fojo, Rockville, MD (US); Susan E Bates, Bethesda, MD (US); Merrill Goldsmith, Bethesda, MD (US); Masaki Kitazono, Kagoshima city (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/487,551

(22) PCT Filed: Aug. 23, 2002
(86) PCT No.: PCT/US02/26908

Related U.S. Application Data
(60) Provisional application No. 60/315,008, filed on Aug. 24, 2001.

(51) Int. Cl. A61K 38/12; A61K 31/19

(52) U.S. Cl. 514/9; 514/575

(57) ABSTRACT

A method of increasing the uptake of an adenoviral agent by a cell, which method comprises contacting the cell with a histone deacetylase inhibitor in an amount sufficient to increase the expression of coxsackie-adenovirus receptors and/or αᵥ integrins on the surface of the cell and subsequently containing the cell with the adenoviral agent, whereupon the uptake of the adenoviral agent by the cell is increased relative to an otherwise identical cell that has not been contacted with a histone deacetylase inhibitor; and a method or preferentially increasing the uptake or an adenoviral agent by a cancerous cell over a normal cell, which method comprises contacting a collection of cells comprising normal cells and a cancerous cell with a histone deacetylase inhibitor i an amount sufficient to increase preferentially the expression of CAR and/or α, integrin on the surface of the cancerous cell over the normal cells and subsequently contacting the collection of cells with the adenoviral agent, whereupon the uptake of the adenoviral agent by the cancerous cell is increased relative to the normal cells.

### FIELD OF THE INVENTION

This invention pertains to a method of using a histone deacetylase inhibitor to increase the entry of an adenoviral agent into a cell by increasing the expression of coxsackie-adenovirus receptor and/or cc, integrins on the surface of the cell.

### BACKGROUND OF THE INVENTION

Adenoviral agents, such as adenoviral vectors, show great promise in the advancement of gene therapy techniques. The construction and use of adenoviral agents for gene delivery is well-known in the art (see, e.g., Young et al., Gut 48:733-736 (2001)).

Adenoviral agents are typically developed using the two most well-known serotypes of adenovirus, serotypes 2 and 5. Current research has indicated that adenovirus serotypes 2 and 5, along with other less characterized adenovirus serotypes, require the coxsackie-adenovirus receptor (CAR) and the αᵥ subunit of the αᵥβ₃ or αᵥβ₅ integrins on the cell surface for efficient infection of the cell to occur (Bergelson et al., Science 275:1320-1323 (1997); Wickham et al., Cell 73:309-319 (1993)). Many cells, however, express low or no levels of the CAR receptor on their cell surfaces. For example, it is believed that one of the reasons why infection by adenoviral agents fails to occur in cancer cells is because most cancer cells have very few or almost none of these receptors on their surfaces (Li et al., Cancer Res. 59:325-330 (1999)).

In order to circumvent low levels of CAR or αᵥ integrin, researchers have employed different strategies to alter the adenovirus so that infection occurs through non-CAR-mediated mechanisms (Krasnykh et al., Cancer Res. 60:6784-6787 (2000)). For example, researchers have blocked the CAR binding site and redirected the adenovirus to the folate receptor (Douglas et al., Nat. Biotechnol. 14:1574-1578 (1996)) or the fibroblast growth factor (FGF) receptor (Goldman et al., Cancer Res. 57:1447-1451 (1997)). See, also, Dmitriev et al., J Virol. 72:9706-9713 (1998)).

While several different strategies have been employed to alter adenoviral agents so that infection occurs through non-CAR-mediated mechanisms, an alternative approach is to increase the levels of cell-surface expression of CAR and/or αᵥ integrin. The present invention seeks to provide such a method whereby the uptake of an adenoviral agent by a cell is increased. This and other objects and advantages of the present invention, as well as additional inventive features, will be apparent from the description of the invention provided herein.

### BRIEF SUMMARY OF THE INVENTION

The present invention provides a method of increasing the uptake of an adenoviral agent by a cell. The method comprises contacting a cell with a histone deacetylase inhibitor in an amount sufficient to increase the expression of CAR and/or αᵥ integrin on the surface of the cell and subsequently contacting the cell with the adenoviral agent, whereupon the uptake of the adenoviral agent by the cell is increased relative to an otherwise identical cell that has not been contacted with a histone deacetylase inhibitor.

The present invention further provides a method of preferentially increasing the uptake of an adenoviral agent by a cancerous cell over a normal cell. The method comprises contacting a collection of cells comprising normal cells and a cancerous cell with a histone deacetylase inhibitor in an amount sufficient to increase preferentially the expression of CAR and/or αᵥ integrin on the surface of the cancerous cell over the normal cells and subsequently contacting the collection of cells with the adenoviral agent, whereupon the uptake of the adenoviral agent by the cancerous cell is increased relative to the normal cells.

### DETAILED DESCRIPTION OF THE INVENTION

The present invention provides a method of increasing the uptake of an adenoviral agent by a cell. The method comprises contacting the cell with a histone deacetylase inhibitor in an amount sufficient to increase the expression of coxsackie-adenovirus receptors and/or αᵥ integrins on the surface of the cell and subsequently contacting the cell with the adenoviral agent, whereupon the uptake of the adenoviral agent by the cell is increased relative to an otherwise identical cell that has not been contacted with a histone deacetylase inhibitor. By “uptake” is meant the process of all or part of an adenoviral agent passing through the membrane of a cell into the interior of the cell, as understood by one skilled in the art. For example, if the adenoviral agent were an adenoviral vector, the nucleic acid of the adenovirus would pass through the cell membrane. By “contacting” is meant exposing the cell to the histone deacetylase inhibitor or adenoviral agent. The cell can be contacted with the histone deacetylase inhibitor or adenoviral agent in any suitable manner, including by in vivo, in vitro and ex vivo methods. Desirably, the cell is first contacted with the histone deacetylase inhibitor and subsequently contacted with the adenoviral agent. Alternatively, the cell is simultaneously contacted with the histone deacetylase inhibitor and the adenoviral agent. By “cell” is meant a single cell or a group of cells, whether isolated, or as part of a tissue, organ or organism. The cell can be normal or abnormal, such as a cancerous cell. Examples of preferred cells include, but are not limited to, bone marrow stem cells, peripheral blood mononuclear cells, peripheral blood stem cells, and vascular endothelial cells. Examples of cancerous cells include, but are not limited to, follicular thyroid, anaplastic thyroid, colon, kidney, breast, and liver cancerous cells. By “organism” is meant an animal, such as a mammal, in particular a human.

The present invention provides for contacting a cell with a histone deacetylase inhibitor. By “histone deacetylase inhibitor” is meant any suitable agent that inhibits an enzyme that removes acetyl groups from proteins, in particular histone proteins. In the present invention, a histone deacetylase inhibitor may include, but is not limited to, known histone deacetylase inhibitors such as depsipeptide (e.g., FR901228, available from Fujisawa Pharma. Co., Ltd., Ibaraki, Japan; Ueda et al., J. Antibiot. (Tokyo) 47:301-310 (1994); Nakajima et al., Exp. Cell Res. 241:126-133 (1998)), sodium butyrate and trichostatin A. Histone deacetylase inhibitors are commercially available (Sigma Chemical Co., St. Louis, Mo.).

By “adenoviral agent” is meant an agent that comprises all or part of an adenovirus. Preferably, the adenoviral agent is a recombinant adenoviral vector comprising a transgene to be expressed in a cell with which it is brought into contact. Alternatively and also preferably, the adenoviral agent comprises one or more adenoviral coat proteins, in particular an adenoviral coat protein that binds to a CAR or αᵥ integrin, in association (e.g., physical or chemical, including, but not limited to, fusion proteins, conjugates and liposomal formulations; see, also, International Patent Application WO 95/21259) with any suitable active agent, such as an agent having a prophylactic (wherein “prophylactic” is intended to encompass prevention and less than complete prevention, such as inhibition of extent of effect or delay of onset of effect) or therapeutic effect (e.g., a pharmaceutical compound, such as a chemotherapeutic agent), whereupon the adenoviral coat protein binds to the CAR or αᵥ integrin and the active agent enters the cell. The use of adenoviral agents is well-known by those ordinarily skilled in the art. See, e.g., Young et al., supra. Adenoviral agents are commercially available. See, e.g., Qbiogene, Carlsbad, Calif.

An adenoviral agent or histone deacetylase inhibitor can be administered to an animal, such as a mammal, in particular a human, in the form of a composition, such as a pharmaceutical composition comprising a pharmaceutically acceptable carrier. Pharmaceutically acceptable carriers are well-known in the art and readily available. The choice of carrier is determined by one of ordinary skill in the art depending on the route of administration, for example.

The amount of histone deacetylase inhibitor with which a cell is brought into contact should be an amount effective in increasing the expression of CAR and/or αᵥ integrin on the surface of the cell. Whether or not an amount of a histone deacetylase inhibitor is effective in increasing the expression of CAR and/or αᵥ integrin can be determined in accordance with the methods of Example 1. The amount of adenoviral agent with which a cell is brought into contact should be an amount effective in achieving a desirable result, e.g., a prophylactic or therapeutic effect. The amount of histone deacetylase inhibitor or adenoviral agent administered to an organism, such as a mammal, in particular a human, in the context of the present invention can be determined by one of ordinary skill in the art. Preferably, the serum level of the histone deacetylase inhibitor, e.g., depsipeptide, is between about 1 ng/ml and about 500 ng/ml. If preferential enhancement of adenoviral transgenic expression in malignant cells over normal cells is desired, preferably the serum level of the histone deacetylase inhibitor, e.g., depsipeptide, is low, such as around 1 ng/ml. If a recombinant adenoviral vector comprising a transgene is used as the adenoviral agent, viral titers as low as one viral particle per cell can be used. These levels can be adjusted up or down depending on the situation.

In view of the foregoing, the present invention provides a method of preferentially increasing the uptake of an adenoviral agent by a cancerous cell over a normal cell. The method comprises contacting a collection of cells comprising normal cells and a cancerous cell with a histone deacetylase inhibitor in an amount sufficient to increase preferentially the expression of CAR and/or αᵥ integrin on the surface of the cancerous cell over the normal cells and subsequently contacting the collection of cells with the adenoviral agent, whereupon the uptake of the adenoviral agent by the cancerous cell is increased relative to the normal cells. Preferably, the amount of histone deacetylase inhibitor administered is low, such as to effect a serum level of around 1 ng/ml.

The present invention further provides another method of increasing the uptake of an adenoviral agent by a cell. The method comprises contacting the cell with a histone deacetylase inhibitor, other than butyrate, in an amount sufficient to increase the expression of CAR and/or αᵥ integrins on the surface of the cell and subsequently contacting the cell with the adenoviral agent, whereupon the uptake of the adenoviral agent by the cell is increased relative to an otherwise identical cell that has not been contacted with a histone deacetylase inhibitor. Preferably, the cell is other than a cancerous bladder cell, such as a bone marrow stem cell, a peripheral blood mononuclear cell, a peripheral blood stem cell, or a vascular endothelial cell. The histone deacetylase inhibitor can be depsipeptide, such as FR901228, or trichostatin A (when the cell is a vascular endothelial cell, in which case depsipeptide is preferred over trichostatin A). The adenoviral agent is as described above.

The present invention further provides yet another method of increasing the uptake of an adenoviral agent by a cell, wherein the cell is other than a cancerous bladder cell. The method comprises contacting the cell with a histone deacetylase inhibitor in an amount sufficient to increase the expression of CAR and/or αᵥ integrins on the surface of the cell and subsequently contacting the cell with the adenoviral agent, whereupon the uptake of the adenoviral agent by the cell is increased relative to an otherwise identical cell that has not been contacted with a histone deacetylase inhibitor. Preferably, the histone deacetylase inhibitor is other than butyrate. Preferably, the cell is a bone marrow stem cell, a peripheral blood mononuclear cell, a peripheral blood stem cell, or a vascular endothelial cell. The histone deacetylase inhibitor can be depsipeptide, such as FR901228, or trichostatin A (when the cell is a vascular endothelial cell, in which case depsipeptide is preferred over trichostatin A). The adenoviral agent is as described above.

### EXAMPLES

The following examples further illustrate the present invention, but should not be construed in any way as limiting its scope.

### Example 1

This example demonstrates that contacting a cancerous cell with a histone deacetylase inhibitor increases the levels of both CAR and αᵥ integrin on the surface of the cell and further results in an increased uptake of an adenoviral agent by the cell.

Cell Lines:

A total of 6 human cancer cell lines were tested: a follicular thyroid carcinoma (FTC 236) (Demeure et al., World J. Surg. 16:770-776 (1992)); an anaplastic thyroid carcinoma (SW-1736) (Ain et al., J. Clin. Endocrinol. Metab. 81:3650-3653 (1996)); a colon carcinoma (SW620) (Leibovitz et al., Cancer Res. 36:4562-4569 (1976)); a renal cell carcinoma (A498) (Giard et al., J. Natl. Cancer Inst. 51:1417-1423 (1973)); a breast carcinoma (MCF7) (Soule et al., J. Natl. Cancer Inst. 51:1409-1413 (1973)); and a hepatocarcinoma (HepG2) (Knowles et al., Science 209:497-499 (1980)).

Adenovirus:

The Ad5.CMV-LacZ is an E1 and E3 gene deleted replication defective type 5 adenovirus obtained from Qbiogene (Carlsbad, Calif.). It was grown in 293A cells according to protocols supplied by the company. The AdCMVβgal virus was purified and the titer was determined by the TCID₅₀ assay as described by the manufacturer.

Depsipeptide:

FR901228 is a depsipeptide fermentation product from Chromobacterium violaceum and was first isolated by the Fujisawa Company (Ueda et al., supra.)

PCR Amplification of CAR and Integrin αᵥ:

RT-PCR for CAR and integrin αᵥ was performed using total RNA extracted with the RNeasy Mini Kit (Qiagen, Valencia, Calif.). Single-stranded oligo(dt)-primed cDNA was generated from 1 μg of RNA in a 20 μl reaction using MMLV reverse transcriptase (Life Technologies, Eggenstein, Germany). Oligonucleotide primers used for analysis of human CAR (Bergelson et al., supra) and αᵥ integrin (Suzuki et al., Proc. Natl. Acad. Sci. U.S.A. 83:8614-8618 (1986)) RNA expression were:

| | |
|---|---|
| CAR 5′ (sense): | ⁴¹⁹GCCTTCAGGTGCGAGATGTTAC⁴⁴⁰ |
| CAR 3′ (antisense): | ¹⁰³¹TCTAAGTCGAATGGGTGCGA¹⁰⁵⁰ |
| Integrin αᵥ 5′ (sense): | ¹⁵⁶⁷TAAAGGCAGATGGCAAAGGAGT¹⁵⁸⁸ |
| Integrin αᵥ 3′ (antisense): | ²⁰³⁶CAGTGGAATGGAAACGATGAGC²⁰⁵⁷ |

These primers generated products that were 631 bp (CAR) and 490 bp (αᵥ integrin) in length. The amplification reaction was carried out with 1 μg of the cDNA product for 30 cycles, and each cycle consisted of 94° C for 20 sec, 64° C. for 30 sec and 72° C. for 1 min, followed by a final 10 min elongation at 72° C. Comparability of RNA quantities was assured using β-actin as an internal standard. Oligonulceotide primers for human β-actin RNA amplification were:

| | |
|---|---|
| β-actin 5′ (sense): | ²⁰⁷TGGGCATGGGTCAGAAGGAT²²⁶ |
| β-actin 3′ (antisense): | ⁴⁸⁸GAGGCGTACAGGGATAGCAC⁵⁰⁷ |

Transduction Efficiency by Ad-βgal With or Without Depsipeptide:

10⁴ untreated cells or 10⁴ cells pre-treated with 1 ng/ml depsipeptide for 72 hrs were plated on a round cover glass (DAIGGER, Vernon Hills, Ill.) in 24 well plates. Cells were transduced with 100 MOI of Ad-βgal in medium without serum for 1 hr and maintained with serum-containing medium for 48 hrs after transduction. Adenovirus transgene expression was compared using the β-Gal Staining Kit (Invitrogen, Carlsbad, Calif.) and β-gal positive cells were counted from three non-overlapping fields.

Protein Collection and Western Blot Analysis:

Supernatants were collected as nuclear extracts. Ten lag of protein were separated on an 11% SDS-PAGE gel, and electroblotting to Immobilon™-P transfer membrane (Millipore, Bedford, Mass.) was performed. The membrane was incubated for 30 min with either a rabbit polyclonal antibody against acetylated histone H3 (Upstate Biotechnology, Lake Placid, N.Y.) or a rabbit polyclonal antibody against histone H3 (Upstate Biotechnology, Lake Placid, N.Y.) diluted 1:2000 in 5% milk. After washing, anti-rabbit Ig horseradish peroxidase-linked secondary antibody (Amersham Pharmacia Biotech, Piscataway, N.J.) was added and incubated for 30 min. After washing, the membrane was developed in ECL™ Western blotting detection reagents (Amersham Pharmacia Biotech, Piscataway, N.J.).

Results

After incubation in 1 ng/ml depsipeptide for 72 hrs, increased expression of endogenous CAR and αᵥ integrin was observed in all cell lines, with similar expression achieved in all of the six cell types. In cells incubated for 72 hrs with 1 ng/ml depsipeptide and then infected with an adenovirus carrying a β-galactosidase gene under the direction of the CMV promoter, 72-88% of cells expressed β-galactosidase with all cell lines having marked increases in the frequency of expression of the β-galactosidase transgene. Incubation in 1 ng/ml depsipeptide resulted in a marked increase in histone acetylation. Similar results were observed with two other histone deacetylase inhibitors, namely sodium butyrate and trichostatin A.

This example illustrates that induction of CAR and a integrin and increased uptake of an adenoviral agent by the cancerous cells occurs due to the action of a histone deacetylase inhibitor, such as depsipeptide.

### Example 2

This example demonstrates that contacting a vascular endothelial cell with a histone deacetylase inhibitor increases the levels of both CAR and αᵥ integrin on the surface of the cell and further results in an increased uptake of an adenoviral agent by the cell.

RT-PCR analysis of CAR and αᵥ integrin RNA levels showed that the histone -deacetylase inhibitor FR901228 increased the level of CAR significantly in human umbilical vein endothelial cells (HUVEC) at a concentration of 0.3 ng/ml for 48 hr.

RT-PCR analysis of CAR and αᵥ integrin RNA levels showed that the histone deacetylase inhibitor trichostatin A increased the level of CAR slightly in HUVEC, while sodium butyrate had little effect.

Western blot analysis of acetylated histone H3 showed that FR901228 increased the level of acetylated histone H3 substantially in HUVEC, while not altering the level of histone H3.

Analysis of an adenoviral vector expressing the β-galactosidase protein showed that expression was observed in less than 5% of the control cells. HUVEC cells treated with 0.3 ng/ml of FR901228 for 48 hr prior to adenoviral infection resulted in over 80% of the cells expressing the β-galactosidase transgene.

### Example 3

This example demonstrates that contacting a normal and an abnormal (i.e., cancerous) hematopoietic cell with a histone deacetylase inhibitor increases the levels of both CAR and αᵥ integrin on the surface of the cell and further results in an increased uptake of an adenoviral agent by the cell.

RT-PCR analysis of CAR and αᵥ integrin RNA levels showed that the histone deacetylase inhibitor FR901228 increased the level of CAR about 3-fold in K562 cells (a cell line derived from a human chronic myeloid leukemia in erythroid blast crisis and available from ATCC, Manassas, Va.) at a concentration of 1 ng/ml. Comparable levels of CAR were observed in G-CSF-mobilized peripheral blood mononuclear cells (PBMNCs; Poietic Technologies, Gaithersburg, Md.) and CD34⁺ selected peripheral blood stem cells (PBSCs; Poietic Technologies) at a concentration of FR901228 of less than 3 ng/ml.

Western blot analysis of acetylated histone H3 showed that FR901228 increased the level of acetylated histone H3 substantially in K562 cells and CD34⁺ PBSCs. No significant change in total histone H3 levels was observed.

Analysis of an adenoviral vector expressing the β-galactosidase protein showed that expression was observed in more than 80% of K562 cells, PBMNCs and CD34⁺ PBSCs at low viral titers (moi=10 for PBMNCs and PBSCs; moi=50 for K562) and short incubation times (i.e., less than 1 hr) when the cells were pre-treated with FR901228 (1 ng/ml for 24 hr for K562; 0.1 ng/ml for 24 hr for PBMNCs and PBSCs).

In addition to the above, PBMNCs were obtained from a patient enrolled in a Phase I depsipeptide (FR901228) study (concentrations used with cultured cells are within the range administered to patients) and examined. CAR expression was found to increase after completion of a 4 hr depsipeptide infusion. A further increase in CAR expression was found at 24 hr of depsipeptide infusion. The level of acetylated histone H3 also increased after administration of depsipeptide.

### Example 4

This example demonstrates that a low concentration of histone deacetylase inhibitor results in the preferential increase in the uptake of an adenoviral agent by a cancerous cell over a normal cell.

A498, MCF7 and HepG2 cancerous cell lines and normal cells (Clonetics, Walkersville, Md.) were treated with FR901228. Treatment with 1 ng/ml FR901228 increased CAR expression in cancerous cells to levels higher than those found in normal cells treated in the same manner. Induction of CAR was only observed in normal cells at 10-20 ng/ml FR901228 and then only a two-fold increase in CAR was observed. The levels of αᵥ integrins in cancerous cells increased from undetectable to a level similar to that found in untreated normal cells. Normal cells treated in the same manner demonstrated little increase in the levels of o integrins. Similarly, increased histone acetylation was observed in cancerous cells treated with 1 ng/ml FR901228, but not normal cells. A higher concentration of FR901228 was required to increase histone acetylation in normal cells. Following FR901228 treatment, a marked increase in β-galactosidase expression upon infection with AdCMBβgal was observed in all cancerous cell lines (4-10 fold with 75-85% of cancerous cells expressing β-galactosidase), but not in normal cells similarly treated.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Of course, variations of those preferred embodiments will become apparent to those of ordinary skill in the art upon reading the foregoing description. The inventors expect ordinarily skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

1. A method of increasing the uptake of an adenoviral agent by a cell, which method comprises contacting the cell with a histone deacetylase inhibitor in an amount sufficient to increase the expression of coxsackie-adenovirus receptors and/or αᵥ integrins on the surface of the cell and, simultaneously with or subsequently to, contacting the cell with the adenoviral agent, whereupon the uptake of the adenoviral agent by the cell is increased relative to an otherwise identical cell that has not been contacted with a histone deacetylase inhibitor.

2. The method of claim 1, wherein the cell is a bone marrow stem cell, a peripheral blood stem cell, or a peripheral blood mononuclear cell.

3. The method of claim 1, wherein the cell is a vascular endothelial cell.

4. The method of claim 1 wherein the cell is cancerous.

5. The method of claim 1, wherein the histone deacetylase inhibitor is depsipeptide.

6. The method of claim 5, wherein the depsipeptide is FR901228.

7. The method of claim 1 wherein the histone deacetylase inhibitor is sodium butyrate.

8. The method of claim 1, wherein the histone deacetylase inhibitor is trichostatin A.

9. The method of claim 1 wherein the adenoviral agent is a recombinant adenovirus that comprises and expresses a transgene.

10. The method of claim 1, wherein the adenoviral agent comprises one or more adenoviral coat proteins in association with an active agent.

11. The method of claim 1, wherein the cell is in vivo.

12. The method of claim 11, wherein the cell is in a mammal.

13. The method of claim 12, wherein the mammal is a human.

14. A method of preferentially increasing the uptake of an adenoviral agent by a cancerous cell over a normal cell, which method comprises contacting a collection of cells comprising normal cells and a cancerous cell with a histone deacetylase inhibitor in an amount sufficient to increase preferentially the expression of CAR and/or αᵥ integrin on the surface of the cancerous cell over the normal cells and subsequently contacting the collection of cells with the adenoviral agent, whereupon the uptake of the adenoviral agent by the cancerous cell is increased relative to the normal cells.

15. The method of claim 14, wherein the histone deacetylase inhibitor is depsipeptide.

16. The method of claim 15, wherein the depsipeptide is FR901228.

17. The method of claim 14, wherein the histone deacetylase inhibitor is sodium butyrate.

18. The method of claim 14, wherein the histone deacetylase inhibitor is trichostatin A.

19. The method of claim 14 wherein the adenoviral agent is a recombinant adenovirus that comprises and expresses a transgene.

20. The method of claim 14, wherein the adenoviral agent comprises one or more adenoviral coat proteins in association with an active agent.

21. The method of claim 14 wherein the cell is in vivo.

22. The method of claim 21, wherein the cell is in a mammal.

23. The method of claim 22, wherein the mammal is a human.

24. The method of claim 21, wherein the amount of histone deacetylase inhibitor administered results in a serum level of around 1 ng/ml.

25. A method of increasing the uptake of an adenoviral agent by a cell, which method comprises contacting the cell with a histone deacetylase inhibitor, other than butyrate, in an amount sufficient to increase the expression of coxsackie-adenovirus receptors and/or αᵥ integrins on the surface of the cell and subsequently contacting the cell with the adenoviral agent, whereupon the uptake of the adenoviral agent by the cell is increased relative to an otherwise identical cell that has not been contacted with a histone deacetylase inhibitor.

26. The method of claim 25, wherein the cell is other than a cancerous bladder cell.

27. The method of claim 25, wherein the cell is a bone marrow stem cell, a peripheral blood stem cell, or a peripheral blood mononuclear cell.

28. The method of claim 25, wherein the cell is a vascular endothelial cell.

29. The method of claim 25, wherein the histone deacetylase inhibitor is depsipeptide.

30. The method of claim 29, wherein the depsipeptide is FR901228.

31. The method of claim 25, wherein the histone deacetylase inhibitor is trichostatin A.

32. The method of claim 25, wherein the adenoviral agent is a recombinant adenovirus that comprises and expresses a transgene.

33. The method of claim 25, wherein the adenoviral agent comprises one or more adenoviral coat proteins in association with an active agent.

34. A method of increasing the uptake of an adenoviral agent by a cell, wherein the cell is other than a cancerous bladder cell, which method comprises contacting the cell with a histone deacetylase inhibitor in an amount sufficient to increase the expression of CAR and/or cc integrins on the surface of the cell and subsequently contacting the cell with the adenoviral agent, whereupon the uptake of the adenoviral agent by the cell is increased relative to an otherwise identical cell that has not been contacted with a histone deacetylase inhibitor.

35. The method of claim 34, wherein the histone deacetylase inhibitor is other than butyrate.

36. The method of claim 34, wherein the cell is a bone marrow stem cell, a peripheral blood stem cell, or a peripheral blood mononuclear cell.

37. The method of claim 34, wherein the cell is a vascular endothelial cell.

38. The method of claim 34, wherein the histone deacetylase inhibitor is depsipeptide.

39. The method of claim 38, wherein the depsipeptide is FR901228.

40. The method of claim 34, wherein the histone deacetylase inhibitor is trichostatin A.

41. The method of claim 34, wherein the adenoviral agent is a recombinant adenovirus that comprises and expresses a transgene.

42. The method of claim 34, wherein the adenoviral agent comprises one or more adenoviral coat proteins in association with an active agent.


SEQUENCE LISTING (6 sequences)

SEQ ID NO: 1 - DNA, 22 bp, Artificial
  Synthetic
gccttcaggtgcgagatgttac

SEQ ID NO: 2 - DNA, 20 bp, Artificial
  Synthetic
tctaagtcgaatgggtgcga

SEQ ID NO: 3 - DNA, 22 bp, Artificial
  Synthetic
taaaggcagatggcaaaggagt

SEQ ID NO: 4 - DNA, 22 bp, Artificial
  Synthetic
cagtggaatggaaacgatgagc

SEQ ID NO: 5 - DNA, 20 bp, Artificial
  Synthetic
tgggcatgggtcagaaggat

SEQ ID NO: 6 - DNA, 20 bp, Artificial
  Synthetic
gaggcgtacagggatagcac